(19) United States
(12) Patent Application Publication
(10) Pub. No.: US 2026/0294768 A1
(43) Pub. Date: Oct. 1, 2026

(54) A PHOTOPROTECTIVE COMPOSITION

(71) Applicant: Conopco, Inc., d/b/a Unilever, Hoboken, NJ (US)

(72) Inventors: Nandini Sachin DABHOLKAR, Mumbai (IN); Anita DAMODARAN, Bangalore (IN); Praful Gulab Rao LAHORKAR, Bangalore (IN); Atul Arunroa MAHAJAN, Amravati (IN); Mruthyunjaya Swamy MATHAPATHI, Bangalore (IN); Amit PATTANAIK, Delhi (IN); Ashish Anant VAIDYA, Bangalore (IN); Satish Kumar VENKATESH, Kolar (IN)

(73) Assignee: Conopco, Inc., d/b/a Unilever, Hoboken, NJ (US)

(21) Appl. No.: 19/100,849

(22) PCT Filed: Aug. 2, 2023
(86) PCT No.: PCT/EP2023/071372

§ 371 Date: Feb. 3, 2025

(30) Foreign Application Priority Data
Aug. 9, 2022 (EP) 22189409.0

(51) Int. Cl. A61K 8/49; A61K 8/34; A61K 8/35; A61K 8/36; A61K 8/37; A61K 8/40; A61K 8/67; A61K 8/73; A61K 8/891; A61Q 17/04

(52) U.S. Cl. CPC A61K 8/4946; A61K 8/345; A61K 8/35; A61K 8/361; A61K 8/37; A61K 8/40; A61K 8/675; A61K 8/676; A61K 8/732; A61K 8/891; A61Q 17/04

(57) ABSTRACT

The present invention relates to a photoprotective personal care composition, which provides high sun-protection factor along with protection against UVA and UVB induced inflammation and oxidative stress. This is achieved through a composition comprising a combination of an oil-soluble sunscreen, a water-soluble sunscreen, a non-ionic surfactant having an HLB value in the range of 9 to 20 along with select vitamins.

### FIELD OF THE INVENTION

The present invention relates to a photoprotective composition, which provides high sun-protection factor. Additionally, the topical composition is so formulated as to deliver high protection against UV induced inflammatory and oxidative stress which could potentially be caused by some amount of UV rays penetrating the sunscreen layer and reaching the skin.

### BACKGROUND OF THE INVENTION

Solar radiation includes about 5% ultraviolet (UV) radiation, wavelength of which is between 200 nm and 400 nm. It is further divided into three regions: from 320 to 400 nm (UV-A), 280 to 320 nm (UV-B) and from 200 to 280 nm (UV-C). Substantial portion of UV-C radiation is absorbed by ozone. Scientific studies have indicated that exposure to UV-A and UV-B radiation for short period causes pigmentation, reddening of the skin and localized irritation, whereas continued or prolonged exposure can lead to sunburn, melanoma, formation of wrinkles and age spots. UV radiation is also thought to cause significant damage to hair. Therefore, it is desirable to protect the skin and other keratinous substrates of the human body from the harmful effects of both UV-A and UV-B radiation. This is delivered though cosmetic compositions which provide high SPF (Sun Protection Factor).

Various cosmetic preparations are reported for preventing and/or protecting the skin from harmful effects of ultraviolet radiation. Numerous organic sunscreen agents capable of absorbing UV-A rays are reported in the field of cosmetics amongst which a particularly useful sunscreen is t-butylmethoxydibenzoylmethane (also known as Avobenzone and sold as Parsol® 1789). A variety of UV-B sunscreens are used in personal care compositions for protection from UV-B radiation. Many cosmetic manufacturers prefer to include UV-A as well as UV-B sunscreens in photoprotective compositions to provide protection over the entire range of UV radiation.

One such patent from the present applicants is published as EP2575748B1 which discloses a composition which delivers high SPF. This publication only discloses use of certain oil soluble sunscreens. The efficacy of sun-protection in this patent is enhanced through inclusion of certain non-ionic surfactants in combination with fatty acid and optionally certain specific polymers. With the advent of many water-soluble sunscreens, the present inventors experimented with a combination of oil-soluble and water-soluble sunscreens in order to deliver better SPF. While the inclusion of oil soluble and water soluble sunscreens ensure high sun protection, there are two problems with this approach. Increasing the amount of such sunscreens does not increase the sun-protection in a proportional manner, rather there is a plateauing of the efficacy. Secondly increasing the amount of sunscreens concomitantly increases the cost. Even at very high sunscreen inclusion there is always a residual amount of UV rays (both UVA and UVB) that penetrates the sunscreen layer which causes inflammation and oxidative stress on the skin. The present inventors on the one hand wish to deliver high sun protection while on the other hand deliver it at as low a cost as possible to the consumer while ensuring that inflammation of the skin and oxidative stress is kept as low as possible.

Additionally, visible light e.g. blue light, is also reported to give rise to unwanted effects e.g. aging, and pigmentation. Therefore, there is also a need to provide compositions that provide protection against visible light e.g. blue light.

The present inventors have achieved this by using both a water soluble as well as an oil soluble sunscreen, use of certain non-ionic surfactants and select vitamins viz. vitamin B3 and vitamin C. The vitamins are shown to interact synergistically in such a composition to deliver high anti-inflammation and protection against oxidative stress.

It is thus an object of the present invention to provide for a photoprotective composition comprising both oil soluble and water-soluble sunscreens that gives high SPF while delivering good protection against UV induced inflammation and oxidative stress.

It is another object of the present invention to provide for a photoprotective personal care composition which gives high SPF and excellent protection against UV induced inflammation and oxidative stress, while delivering the desired sensorial attributes on the skin.

It is another object of the present invention to provide a photoprotective personal care composition which provides protection against visible light induced e.g. blue light induced, oxidative stress.

It is another object of the present invention to provide a photoprotective personal care composition which provides protection against visible light e.g. blue light, induced oxidative stress, while delivering the desired sensorial attributes on the skin.

### SUMMARY OF THE INVENTION

The first aspect of the present invention relates to a photoprotective composition comprising:
(i) 0.1 to 10 wt % oil-soluble sunscreen;
(ii) 0.2 to 5 wt % water-soluble sunscreen;
(iii) 0.1 to 5 wt % non-ionic surfactant having an HLB value in the range of 9 to 20;
(iv) 0.1 to 3 wt % vitamin B3 compound, a derivative or precursor thereof;
(v) 0.01 to 5 wt % vitamin C or derivative thereof.
wherein, the composition additionally comprises 3 to 25 wt % one or more saturated fatty acids.

Another aspect of the present invention relates to a method of providing photoprotection with a Sun Protection Factor (SPF) of at least 15 comprising a step of applying a composition of the first aspect on to the desired skin surface.

Yet another aspect of the present invention relates to a method of protecting skin against UVA and UVB induced inflammation and oxidative stress comprising the step of applying a composition of the first aspect on to the desired skin surface.

Yet another aspect of the present invention relates to a method of protecting skin against visible light e.g. blue light, induced oxidative stress comprising the step of applying a composition of the first aspect on to the desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The photoprotective composition of the invention is meant to be used for personal care or for cosmetic use and could also be referred to as a personal care composition or a cosmetic composition. By a “personal care composition” as used herein, is meant to include a composition for topical application to sun-exposed areas of the skin and/or hair of humans. Such a composition may be classified as leave-on or rinse off, and includes any product applied to a human body for improving appearance, cleansing, odour control or general aesthetics. The composition of the present invention can be in the form of a liquid, lotion, cream, foam, stick or gel. Non-limiting examples of such sunscreen compositions include leave-on skin lotions, creams, antiperspirants, deodorants, foundations, mascara, sunscreen lotions and wash-off shampoos, conditioners and shower gels. The composition of the present invention is preferably a leave-on composition.

“Skin” as used herein is meant to include skin on the face and body (e.g., neck, chest, back, arms, underarms, hands, legs and scalp) and especially to the sun exposed parts thereof. Compositions of the invention is also of relevance to applications on any other keratinous substrates of the human body other than skin e.g. hair where products may be formulated with specific aim of improving photoprotection.

The composition of the invention comprises 0.1 to 10% by weight oil-soluble sunscreen. By oil-soluble is meant those organic sunscreens which have a solubility in water of less than 10 g/l, preferably less than 5 g/l, more preferably less than 2 g/l. Thus, most oil-soluble sunscreens for use in the present invention has a solubility in water of 0 to 2 g/l. Oil-soluble sunscreens could be of the UV-B type or of the UV-A type. It is preferred that compositions in accordance with the invention comprise 1 to 8% by weight of such sunscreens.

When an oil soluble UVB sunscreen is included, it is preferably one or more of octyl salicylate, 3,3,5-trimethylclohexyl 2-hydroxybenzoate, ethylhexyl salicylate, 2-ethylhexyl 2-cyano-3,3-diphenyl-2-propenoate which is also known as octocrylene, 2-ethylhexyl-4-methoxycinnamate or octylmethoxycinnamate, ethylhexyl triazone, 3-benzylidene camphor, cinoxate, diethylhexyl butamido triazone, para amino benzoic acid (PABA), ethyl dihydroxypropyl PABA, ethylhexyl dimethoxy benzylidene dioxoimidazoline propionate, isoamyl p-methoxycinnamate, isopentyl trimethoxycinnamate trisiloxane, isopropyl benzyl salicylate, isopropyl methoxycinnamate, 4-methylbenzylidene camphor, PEG-25 PABA, and pentyl dimethyl PABA. More preferred oil-soluble UVB sunscreens are selected from one or more of octyl salicylate, 3,3,5-trimethylclohexyl 2-hydroxybenzoate, ethylhexyl salicylate, 2-ethylhexyl 2-cyano-3,3-diphenyl-2-propenoate, and 2-ethylhexyl-4-methoxycinnamate and further more preferred are selected from one or more of 2-ethylhexyl-4-methoxycinnamate, ethylhexyl salicylate and 2-ethylhexyl 2-cyano-3,3-diphenyl-2-propenoate. Some of the well known brands under which the above sunscreens are sold are Octisalate®, Homosalate®, Neo Heliopan®, Neo Heliopan® AV, Neo Heliopan® OS, Octocrylene® and Parsol® MCX. The oil-soluble UV-B sunscreen has Amax from 280 to 320 nm.

Composition of the invention may comprise an organic oil-soluble UVA sunscreen. When included, it is selected from one or more of a dibenzoylmethane compound, diethylamino hydroxybenzoyl hexyl benzoate and methyl anthranilate. A dibenzoyl methane compound is most preferred for an oil-soluble UVA-sunscreen. Suitable dibenzoyl methane compounds are selected from one of more of t-butylmethoxy dibenzoylmethane, 2-methyldibenzoylmethane, 4-methyl-dibenzoyl-ethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane and 2,6-dimethyl-4-tert-butyl-4′-methoxy-dibenzoylmethane. The most preferred dibenzoyl methane compound is t-butylmethoxy dibenzoylmethane. Commercially available as Parsol 1789.

Composition of the invention may comprise both an organic oil-soluble UVB and an organic UVA sunscreen. Alternately, there are certain oil soluble sunscreens which have both a UVA and UVB screening efficacy. It is selected from one or more bis-ethylhexyloxyphenol methoxyphenyl triazine, drometrizole trisiloxane, benzophenone and its derivatives. The most preferred one is bis-ethylhexyloxyphenol methoxyphenyl triazine, commercially available as Tinosorb S.

The composition of the invention comprises 0.2 to 5 wt % of a water soluble sunscreen. By water soluble sunscreen, whether of the UVA type, or of the UVB type, is meant that the solubility in water of the sunscreen is higher than 10 g/l preferably higher than 50 g/l at 25° C. Most water soluble sunscreens for use in the present invention have a water solubility in the range of 10 to 100 g/l.

The preferred water-soluble UVB sunscreen for use in the present invention is phenyl benzimidazole sulphonic acid (PBSA), benzophenone-4 or benzylidene camphor sulfonic acid, preferably PBSA. PBSA also known as Ensulizole is commercially available as Eusolex 232 (from Merck KGaA). PBSA is also available under the brand names Neo Heliopan Hydro (from Symrise), Parsol HS (from DSM) and Sunsafe ES (from Uniproma). There are certain water soluble sunscreens which have both a UVA screening efficacy as well as a UVB screening efficacy. Such sunscreens provide both UVA and UVB protection e.g. benzophenone-4 (sold as Sulisobenzone). However for the purposes of the present invention benzophenone-4 may be included as a water-soluble UV-B sunscreen.

The preferred UVA sunscreen for use in the present invention is one or more of disodium phenyl dibenzimidazole tetrasulfonate (Neoheliopan AP) or terephthalylidene dicamphor sulfonic acid (TDSA). Di sodium phenyl dibenzimidazole tetra sulfonate is also known as bisdisulizole disodium. This is commercially available as Neo heliopan AP (from Symrise Shanghai Ltd) or as Sunsafe DPDT (from Uniproma)

Terephthalylidene dicamphor sulfonic acid (TDSA) is also known as Ecamsule. This is commercially available as Mexoryl SX (U.S. Pat. No. 4,585,597) by L′ Oreal or Sunsafe TDSA (from Uniproma). It can be used as parent acid or its salts to deliver the desired benefit. The composition preferably comprises 0.1 to 5 wt %, more preferably 1 to 3 wt % water soluble UVA sunscreen. The composition preferably comprises 0.1 to 5 wt %, more preferably 1 to 4 wt %, water soluble UVB sunscreen.

The water-soluble sunscreens for inclusion in the composition of the present invention are generally commercially available in the acid form. When included in the composition in the acid form (pre neutralized forms) i.e having sulphonic acid group (—SO₃H) the composition additionally comprises a neutralising agent to convert the acid form into the salt form, in which form it is known to be active as a sunscreen with an exception to Mexoryl SX which can act as sunscreen with and without neutralization. When included, the neutralising agent is preferably in 0.05% to 4% more preferably 0.2% to 2% by weight of the composition. The neutralising agent is preferably an inorganic or an organic alkali. Organic alkali is preferably an amine such as triethanol amine or diethanol amine. The present inventors have observed that the inorganic alkali is especially preferred. Preferred are alkali metal hydroxides. Most preferred metal hydroxide for inclusion as neutralising agent in the composition of the invention are sodium hydroxide or potassium hydroxide.

The composition of the invention comprises a non-ionic surfactant having an HLB value in the range of 9 to 20. More preferably the nonionic surfactant is selected from those having HLB value in the range 12 to 20, further more preferably 12 to 18.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20.

Preferably, the nonionic surfactant having HLB value in the range 9 to 20 is selected from one or more of fatty alcohol ethoxylates, fatty acid ethoxylate, alkyl phenol ethoxylates, and polyoxyethylene sorbitan alkyl esters.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

Fatty acid ethoxylates are available under the Myrj class. Suitable examples of commercially available non-ionic surfactants for use in the composition of this class are Myrj S40 (PEG-40 stearate), Myrj S50 (PEG-50 stearate), or Myrj 59 (PEG-100 stearate).

Preferably, the composition comprises 0.2 to 5 wt %, more preferably 0.5 to 4 wt %, even more preferably from 1 to 3 wt % nonionic surfactant having HLB in the range 9 to 20.

The composition of the invention includes a vitamin B3 compound, a derivative or precursor thereof. This includes compounds that are carboxylic acid functionalized heteroaromatic compounds. These may be selected from one or more of nicotinic acid, picolinic acid, nicotinate, niacinamide, and picolinamide. It is preferably one or both of niacinamide and picolinamide, most preferably niacinamide. The vitamin B3 compound is typically included in an amount of 0.1 to 5%, preferably, 0.5 to 3% by weight of the composition.

The composition comprises 0.01 to 5 wt % of a vitamin C or a derivative thereof. Vitamin C (ascorbic acid, ascorbate) is a simple low-molecular-weight carbohydrate that is essential for the body as a water-soluble vitamin. Vitamin C has both oxidized and reduced forms: L-dehydroascorbic and L-ascorbic acid. Vitamin C is involved in the formation of the skin barrier and collagen in the dermis and plays a physiological role in the skin against skin oxidation, in antiaging of wrinkles, and in cell signal pathways of cell growth and differentiation, which are related to the occurrence and development of various skin diseases.

Vitamin C or its derivatives which may be used in the present invention may be one or more of the following:

### Sodium Ascorbyl Phosphate:

Sodium ascorbyl phosphate is an active stable vitamin C derivative ingredient for the cosmetics industry. It liberates vitamin C in the skin and protects the cells of the skin, promotes collagen formation, controls the formation of senile keratosis, and lightens dark skin.

### 3-O-Ethyl Ascorbic Acid:

3-O-ethyl ascorbic acid promotes the production of collagen, which significantly improves the structure of the skin cells and the overall condition of skin.

### Ascorbyl Methylsilanol Pectinate:

It is a known antioxidant and is known to improve skin texture, as well as helping with reducing aging and dark spots.

### Magnesium Ascorbyl Phosphate (MAP):

Magnesium ascorbyl phosphate (MAP) has all the functions of vitamin C and is very stable. It has excellent anti-oxidation and protection benefits, and can effectively resist UV radiation and promotes collagen production. This product is recommended for skin whitening/lightening applications, as well as anti-aging and anti-wrinkle products.

### Ascorbic Acid:

Vitamin C (L-ascorbic acid), USP is an active form of vitamin C as it occurs naturally. It is a potent antioxidant (shown to be able to protect skin from oxidative damages). It can improve appearance of aged and fragile skin. It is widely used as an add-on ingredient in skin-lightening products to correct hyperpigmentation and age spots.

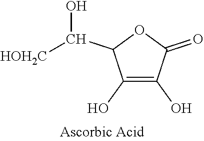 [SMILES: O=C1OC(C(O)CO)C(O)=C1O]

### Ascorbyl Glucoside:

Ascorbyl glucoside is a water-soluble Vitamin C derivative with superior stability. It resists degradation, and has the same lightening, sun protective and anti-aging properties as ascorbic acid.

### Tetrahexyldecyl Ascorbate:

Tetrahexyldecyl ascorbate is a very stable, oil-soluble Vitamin C ester but has no inherent capabilities as an antioxidant because all hydroxyl groups are esterified. It has excellent skin penetration and as a result offers increased cell protection against UV-B radiation. It can improve appearance of aged and fragile skin. It is widely used as an add-on ingredient in skin-lightening products to correct hyperpigmentation and age spots.

### Aminopropyl Ascorbyl Phosphate:

It is a stabilized vitamin C derivative. It is ideal for whitening, anti-wrinkle, and anti-oxidation applications.

Vitamin C or its derivative is preferably included in 0.01 to 2%, more preferably 0.05 to 1.5% by weight of the composition. Vitamin C is preferably included in the composition in encapsulated form. Vitamin C or its derivative, most suitable for use in the present invention, is sodium ascorbyl phosphate. It is available from BASF as SAP, from DSM, as Stay C50, from Vivimed as sodium ascorbyl phosphate, from Uniproma as PromaCare SAP and from Chemspec Chemicals Pvt. Ltd as sodium ascorbyl phosphate.

The composition comprises 3 to 25 wt % one or more saturated fatty acid. The one or more saturated fatty acid is preferably a C10 to C22 fatty acid, more preferably a C16 to C18 fatty acid. Most preferably the one or more saturated fatty acids are stearic acid or palmitic acid or a mixture thereof. The one or more saturated fatty acid is often hystric acid which is substantially (generally about 90 to 95%) a mixture of stearic acid and palmitic acid. Preferred hystric acid are ones having 40-65% saturated C16 and 34-58% saturated C18; or ones having 52-58% saturated C16 and 40 to 47% saturated C18 fatty acids. It is particularly preferred that the composition comprises 4 to 23%, more preferably 5 to 20%, even more preferably 6 to 17%, further more preferably 8 to 15%, still more preferably from 10 to 12% one or more saturated fatty acid by weight of the composition. Without wishing to be bound by theory the inventors believe that the inclusion of one or more saturated fatty acids enhances skin-hydration by sealing in the skin's moisture and reducing evaporation to the surrounding atmosphere. Apart from this benefit it is also seen to boost the SPF through uniformly distributing the oil soluble sunscreen on the skin.

The composition may optionally comprise 0.4 to 10 wt % humectant. Humectants of the polyhydric alcohol-type can be employed as cosmetically acceptable carriers. Preferably the humectant is at least one of propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, glycerol, ethoxylated glycerol, and propoxylated glycerol. Preferred humectants are selected from one or more of glycerol, sorbitol, propylene glycol and 1,3-butylene glycol. Glycerol is the most preferred humectant. It is preferred that the amount of humectant is 1 to 10%, more preferably 2 to 8% by weight of the composition. Without wishing to be bound by theory, the inventors believe that the inclusion of humectant provides hydration benefit. Apart from this, it is seen to boost the SPF by keeping the water soluble sunscreen in the dissolved form.

The composition of the invention may further optionally comprise 0.25 to 5 wt % of a polymer of (a) dimethicone/vinyldimethicone crosspolymer and (b) silica. The dimethicone/vinyldimethicone crosspolymer could be with or without silica. Various polymers of the above types could be used in the present invention. Dimethicone/vinyl dimethicone crosspolymer which is a preferred polymer as per the invention is available as KM-9729, DOWSIL™ EP-9215, SeraSnowSP 13, DOWSIL™9506 Powder, SpecsilSPD. An even more preferred polymer as per the invention is Dimethicone/Vinyl Dimethicone Crosspolymer (and) Silica which is available as DOWSIL™9701 Cosmetic Powder. Other suitable polymers which may be used are listed below:

Dimethicone/Vinyl Dimethicone Crosspolymer (and) Silica (and) Butylene Glycol available as DOWSIL™ EP-9801 Hydro Cosmetic Powder

Dimethicone (and) Dimethicone/Vinyl Dimethicone Crosspolymer (and) Dimethicone Crosspolymer (and) Beeswax (and) Silica (and) Silica Silylate available as DOWSIL™9576 Smooth Away Elastomer

Dimethicone/Vinyl Dimethicone Crosspolymer (and) Polymethylsilsesquioxane (and) Silica Dimethyl Silylate Available as SSP100

Dimethicone/Vinyl Dimethicone Crosspolymer (and) LauroylLysine available as DOWSIL™ EP-9289 LL

Hydrophobically modified silica is preferably selected from Silica Silylate available as DOWSIL™ VM-2270 AerogelFine Particles, HDK® H2000 and

Silica dimethyl silylate available as HDK® H15, HDK® H20, HDK® H30, HDK® H18

The polymer is included in 0.25 to 5%, preferably 0.5 to 4%, more preferably 1 to 3% by weight of the composition.

Without wishing to be bound by theory the inventors believe that the inclusion of polymer provides reduced-shine and mattifying effect on the skin with sensory benefits.

The composition of the invention may additionally comprise a modified starch preferably the modified starch is aluminium starch octenylsuccinate. Aluminum Starch Octenylsuccinate is a chemically modified starch, in particular starches that have undergone esterification. Such aluminum starch Octenylsuccinate is commercially available from suppliers like Akzo Nobel or Nouryon under the names Dry Flo® Pure and Dry Flo® PC. A variety of other suppliers sell it under their own brand name. In cosmetics and personal care products, Aluminum Starch Octenylsuccinate is used in the formulation of lotions, powders, makeup and underarm deodorants. It is used as a thickener and binder, anticaking agent and oil absorbent. The composition may comprise aluminium starch octenylsuccinate which is further compounded with other ingredients to deliver other benefits. e.g. the ingredient having INCI name Aluminum Starch Octenylsuccinate (and) Acrylates Copolymer (and) Magnesium Carbonate may be included. It is available from Nouryon as NATRASORB® HFB. The natural content is 81% and the suggested applications include moisture resistant products, oil control products, anhydrous products, antiperspirant sticks, sunscreen, and dry shampoo for a variety of benefits including oil-absorption, mitigation of syneresis and fragrance-retention. The literature of the ingredient from Nouryon discloses that it is crosslinked aluminium starch octenyl succinate surface treated with acrylates copolymer and magnesium carbonate. The modified starch is preferably included in 0.5 to 10%, preferably 0.5 to 5%, by weight of the composition. Of these, the polymer, when included is preferably at 0.2 to 3%, the silica microsphere when included is from 0.2 to 3% and the modified starch when included is from 0.5 to 4% by weight of the composition. Without wishing to be bound by theory the inventors believe that the inclusion of modified starch provides the following benefits: sebum and oil absorption, smooth and powdery feel along with good spreadability and slipperiness while boosting SPF through uniform distribution of the sunscreen.

Water is preferably included in 35 to 90%, more preferably 50 to 85%, further more preferably 50 to 80% by weight of the composition.

In addition to the ingredients disclosed earlier, the compositions, may and preferably do include other ingredients in order to perform one or more functions of this invention also include a cosmetically acceptable carrier. Emollient materials may serve as cosmetically acceptable carriers. These may be in the form of silicone oils, natural or synthetic esters, and hydrocarbons. Amounts of the emollients may range anywhere from 0.1 to 25%, preferably between 1 and 10% by weight of the composition. Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 5 to 6, silicon atoms.

Nonvolatile silicone oils useful as an emollient materials include polyalkyl siloxanes, polyalkylaryl siloxanes and polyether siloxane copolymers. The essentially nonvolatile polyalkyl siloxanes useful herein include, for example, polydimethyl siloxanes with viscosities of from 5×10⁻⁶ to 0.1 m²/s at 25° C. Among the preferred nonvolatile emollients useful in the present compositions are the polydimethyl siloxanes having viscosities from 1×10⁵ to 4×10⁴ m²/s at 25° C.

Another class is of ester emollients, which are alkyl esters of saturated fatty acids having 10 to 24 carbon atoms. Examples thereof include behenyl neopentanoate, isononyl isonanonoate, isopropyl myristate and octyl stearate. Alternatively, they are ether-esters such as fatty acid esters of ethoxylated saturated fatty alcohols. Further alternatively they are polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C1-C30 alcohols. Yet further alternatively they are wax esters such as beeswax, spermaceti wax and tribehenin wax. Yet further alternatively they are sugar esters of fatty acids such as sucrose polybehenate and sucrose polycottonseedate. Further alternatively they are natural ester emollients principally based upon mono-, di- and tri-glycerides. Representative glycerides include sunflower seed oil, cottonseed oil, borage oil, borage seed oil, primrose oil, castor and hydrogenated castor oils, rice bran oil, soybean oil, olive oil, safflower oil, shea butter, jojoba oil and combinations thereof. Animal derived emollients are represented by lanolin oil and lanolin derivatives. Amounts of the natural esters may range from 0.1 to 20% by weight of the compositions.

Hydrocarbons, which are suitable cosmetically acceptable carriers include petrolatum, mineral oil, C₈-C₁₈ isoparaffins, polybutenes and especially isohexadecane, available commercially as Permethyl® 101A from Presperse Inc.

Fatty alcohols having from 10 to 30 carbon atoms are another useful category of cosmetically acceptable carrier. Illustrative of this category are stearyl alcohol, lauryl alcohol, myristyl alcohol, oleyl alcohol and cetyl alcohol and mixtures thereof. Thickeners can be utilized as part of the cosmetically acceptable carrier of compositions according to the present invention. Typical thickeners include crosslinked acrylates (e.g. Carbopol® 982 and Carbopol® Ultrez 10), hydrophobically-modified acrylates (e.g. Carbopol 1382 (R)), polyacrylamides (e.g. Sepigel® 305), acryloylmethylpropane sulfonic acid/salt polymers and copolymers (e.g. Aristoflex® HMB and AVC), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminum silicate (Veegum®). Amounts of the thickener may range from 0.0001 to 10%, usually from 0.001 to 2.5%, optimally from 0.01 to 1% by weight of the composition. Preferred are emollients that can be used, especially for products intended to be applied to the face, to improve sensory properties and are chosen from the group of oils that do not form stiff gels with 12HSA; these include polypropylene glycol-14 butyl ether otherwise known as Tegosoft® PBE, or PPG15 stearyl ether such as Tegosoft® E, other oils such as esters, specifically, isopropyl myristate, isopropyl palmitate, other oils could include castor oils and derivatives thereof.

The inventive compositions preferably includes a skin brightening compound. Illustrative substances are placental extract, lactic acid, arbutin, kojic acid, ferulic acid, and hydroquinone. More preferably, such skin brightening compound is a tyrosinase inhibitor, to complement the melanogenesis inhibition activity of the substituted monoamines, most preferably a compound selected from the group consisting of kojic acid, or hydroquinone. Also, dicarboxylic acids represented by the formula HOOC-(CxHy)-COOH where x=4 to 20 and y=6 to 40 such as azelaic acid, sebacic acid, oxalic acid, succinic acid, fumaric acid, octadecenedioic acid or their salts or a mixture thereof, most preferably fumaric acid or salt thereof, especially di-sodium salt.

Combination of 12HSA with fumaric acid or salts thereof are particularly preferred, especially for skin brightening formulations. Amounts of these agents may range from 0.1 to 10%, preferably from 0.5 to 2% by weight of the composition.

Colorants, opacifiers and abrasives may also be included in compositions of the present invention. Each of these substances may range from 0.05 to 5%, preferably between 0.1 and 3% by weight of the composition.

Preservatives could be incorporated into the compositions of this invention to protect against the growth of potentially harmful microorganisms. Suitable traditional preservatives for compositions of this invention are alkyl esters of para-hydroxybenzoic acid. Other preservatives which have come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, caprylyl glycol, C1-6 parabens (especially, methyl paraben and/or propyl paraben), imidazolidinyl urea, sodium dehydroacetate and benzyl alcohol. The preservatives should be selected having regard for the use of the composition and possible incompatibilities between the preservatives and other ingredients in the emulsion.

Preservatives are preferably employed in amounts ranging from 0.01% to 2% by weight of the composition, including all ranges subsumed therein. An especially preferred combination is octocrylene and caprylyl glycol, since caprylyl glycol has been disclosed to enhance UVA and UVB protection. The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include antioxidants, binders, biological additives, buffering agents, colorants, astringents, fragrance, opacifying agents, conditioners, exfoliating agents, pH adjusters, natural extracts, essential oils, skin sensates, skin soothing agents and skin healing agents.

The compositions of the present invention are preferably non-solid. The compositions of the invention are preferably leave-on compositions. Such compositions are applied on the skin and remain thereon for prolonged time. Rather, as leave-on compositions are not intended to be rinsed-off they need to be non-irritating and therefore it would be necessary to minimize the total level of surfactant and the total level of anionic surfactant in leave-on compositions. The total level of anionic surfactant in the inventive compositions is preferably no more than 10%, more preferably below 8%, most preferably at most 5%, optimally at most 3%.

The compositions of the present invention are preferably in a lotion format. The composition of the present invention provides Sun Protection Factor (SPF) of at least 15.

### Method of Using Compositions

The composition according to the invention is intended primarily as a product for topical application to human skin, especially as an agent for protecting from solar radiation, and preventing or reducing pigmentation, the appearance of wrinkled or aged skin, or age spots.

In use, a small quantity of the composition, for example from 1 to 5 ml, is applied to exposed area of the skin, from a suitable container or applicator and, if necessary, it is then spread over and/or rubbed into the skin using the hand or fingers or a suitable device.

In accordance with another aspect is disclosed a method of providing photoprotection comprising a step of applying a composition of the invention said method provides Sun Protection Factor of at least 15, preferably at least 20, more preferably at least 25 ideally at least 30.

The individual Sun Protection Factor (SPF) for each subject is defined as the ratio of the amount of energy (dose, in units of J/m² or in seconds of exposure time) required to produce minimal erythema on protected skin to the amount of energy needed to produce minimal erythema on untreated skin calculated as follows:

$$SPF = \frac{MED Protected Skin}{MED Unprotected Control Site}$$

The SPF for the product is calculated by taking the arithmetic mean of the individual SPF values for all the subjects.

Yet another aspect of the present invention relates to a method of protecting skin against UVA and UVB induced inflammation and oxidative stress comprising the step of applying a composition of the first aspect on to the desired skin surface. Preferably, the method is non-therapeutic/cosmetic in nature.

Yet another aspect of the present invention relates to a method of protecting skin against visible light e.g. blue light, induced oxidative stress comprising the step of applying a composition of the first aspect on to the desired skin surface. Preferably, the method is non-therapeutic/cosmetic in nature.

While the above summarizes the present invention, it will become apparent to those skilled in the art that modifications, variations and alterations may be made without deviating from the scope and spirit of the present invention as described and claimed herein. The invention will now be further illustrated in the following non-limiting examples.

### EXAMPLES

### Examples A to C and 1: Compositions Tested for SPF and for Anti-Inflammation and Antioxidant Benefit

The following compositions as shown in Table-1 were prepared.

TABLE 1

| | Example | Example | Example | Example |
|---|---|---|---|---|
| Ingredient | A, wt % | B, wt % | C, wt % | 1, wt % |
| Glycerin (98%) | 2.5 | 2.5 | 2.5 | 2.5 |
| Ultrez 10 | 0.5 | 0.5 | 0.5 | 0.5 |
| Glycol Stearate 97% + Stearamide | 1.39 | 1.39 | 1.39 | 1.39 |
| AMP 3% | | | | |
| Glycol Stearate (50% monostearate, | 0.65 | 0.65 | 0.65 | 0.65 |
| low glyceol) | | | | |
| Myrj59 | 1.50 | 1.50 | 1.50 | 1.50 |
| Stearic acid | 6.00 | 6.00 | 6.00 | 6.00 |
| Potassium hydroxide | 0.54 | 0.54 | 0.54 | 0.54 |
| Parsol 1789 | — | — | 2.00 | 2.00 |
| Octyl Salicylate | — | — | 5.00 | 5.00 |
| Parsol HS | — | — | 1.50 | 1.50 |
| Octrocrylene | — | — | 2.00 | 2.00 |
| MT700Z | 0.60 | 0.60 | 0.60 | 0.60 |
| DC200/50cst | 1.75 | 1.75 | 1.75 | 1.75 |
| Dryflo Pure (Aluminium Starch | 1.00 | 1.00 | 1.00 | 1.00 |
| Octenylsuccinte) | | | | |
| Stay C50 | — | 0.25 | — | 0.25 |
| Niacinamide | — | 1.25 | — | 1.25 |
| DC9701 | 1.00 | 1.00 | 1.00 | 1.00 |
| Cetyl alcohol | 0.37 | 0.37 | 0.37 | 0.37 |
| Caprylyl Glycol | 0.50 | 0.50 | 0.50 | 0.50 |
| Sodium hydroxide | 0.23 | 0.23 | 0.23 | 0.23 |
| Xanthan Gum | 0.25 | 0.25 | 0.25 | 0.25 |
| Water | To 100 | To 100 | To 100 | To 100 |

In the table above,

Stay C50 is sodium ascorbyl phosphate sourced from DSM.

The above samples were used for measuring the SPF and UVAPF values using the following procedures:

Thin film transmittance measurements were done using Labsphere's Ultraviolet transmittance analyzer. In this study, 70×70 mm PMMA plate with ~6 μm roughness from Schönberg GmbH & Co were used. The percent transmittance of the various compositions was measured using a procedure as outlined below. 2 mg/cm² of sample was applied on PMMA plate, distributed uniformly using a syringe, and spread uniformly. The drying time for the PMMA plates was 30 minutes. After the drying time, sample plates were exposed to UV light and transmittance scan was recorded. This scan gives the transmittance as a function of wavelength (290-400 nm) for a given sample. For a single plate six to nine different spots were scanned. The same was repeated for 2 or 3 plates. The data reported is thus an average over 12-18 readings. The reference transmittance scan was obtained using a blank plate, with no sample on the PMMA plates with glycerine spread on it. The transmittance values were used to arrive at the SPF and UVA PF values using the UV-2000s application provided with the instrument.

Percentage of transmittance (and therefore the SPF) was measured invitro SPF at zero time (line 0 min). Same plate was exposed to solar simulated light and % of transmittance measured at designated time intervals from 0 and 60 min. Average absorbance at 310 & 355 nm was recorded to measure % UVB & UVA photostability respectively. UV was generated using solar simulator (Atlas kW system) using irradiance of 550 W m⁻², at a distance of ~20 cm.

The SPF and UVAPF values are summarised in Table-2 below:

TABLE 2

| | Example | Example | Example | Example |
|---|---|---|---|---|
| Values measured | A, wt % | B, wt % | C, wt % | 1, wt % |
| SPF | 1.85 | 1.87 | 58.3 | 51.3 |
| SPF (std dev) | — | — | 13.2 | 9.6 |
| UVAPF | 2.37 | 2.40 | 28.1 | 22.3 |
| UVAPF (std dev) | 0.12 | 0.1 | 5.4 | 5.1 |
| Photostability | — | — | ~89% | ~90% |

The data in the table above indicates that one obtains an SPF and UVAPF value for a composition as per the invention (Example 1) which is of similar magnitude as a composition as per an invention having same amount of sunscreen but without the vitamins (B3 and C) i.e. Example C.

Additionally Examples C1 and C2 were also prepared where either Vitamin B3 was included or Vitamin C was included but not both. The compositions are given in the Table-3 below:

TABLE 3

| | Example | Example |
|---|---|---|
| Ingredient | C1, wt % | C2, wt % |
| Glycerin (98%) | 2.5 | 2.5 |
| Ultrez 10 | 0.5 | 0.5 |
| Glycol Stearate 97% + Stearamide | 1.39 | 1.39 |
| AMP 3% | | |
| Glycol Stearate (50% monostearate, | 0.65 | 0.65 |
| low glyceol) | | |
| Myrj59 | 1.50 | 1.50 |
| Stearic acid | 6.00 | 6.00 |
| Potassium hydroxide | 0.54 | 0.54 |
| Parsol 1789 | 2.00 | 2.00 |
| Octyl Salicylate | 5.00 | 5.00 |
| Parsol HS | 1.50 | 1.50 |
| Octrocrylene | 2.00 | 2.00 |
| MT700Z | 0.60 | 0.60 |
| DC200/50cst | 1.75 | 1.75 |
| Dryflo Pure (Aluminium Starch | 1.00 | 1.00 |
| Octenylsuccinte) | | |
| Stay C50 | 0.25 | — |
| Niacinamide | — | 1.25 |
| DC9701 | 1.00 | 1.00 |
| Cetyl alcohol | 0.37 | 0.37 |
| Caprylyl Glycol | 0.50 | 0.50 |
| Sodium hydroxide | 0.23 | 0.23 |
| Xanthan Gum | 0.25 | 0.25 |
| Water | To 100 | To 100 |

The samples of Examples A to C, C1, C2 and Example 1 were subjected to UVA radiation and UVB radiation in separate experiments and the anti-inflammatory benefit (IL1a levels) and antioxidant benefit (ROS levels) were measured using the protocol given below:

Primary skin keratinocytes were seeded in cell culture plates and incubated for 48 h in 5% CO₂ incubator at 37° C. Then cells were exposed to UVA (378 mJ/cm²) or UVB (15 mJ/cm²) by covering PMMA plates on top of the culture plates with and without applying placebo/test formulations on PMMA plates. Bio-actives (Vit B3 at 0.0125% and SAP at 0.0025%), either single or in combinations were added to the cells in the media after UV exposure. Then culture plates were incubated in 5% CO₂ incubator at 37° C. for 6 h. Post 6 h incubation, ROS levels were measured in the cells by DCFDA assay and IL1a was estimated in the supernatant by using IL1a ELISA kit.

The data is summarised in Table-4 and 5 below:

TABLE 4

| | Ex- | Ex- | Ex- | Ex- | Ex- | Ex- |
|---|---|---|---|---|---|---|
| | am- | am- | am- | am- | am- | am- |
| Values | ple | ple | ple | ple | ple | ple 1, |
| measured | A | B | C1 | C2 | C | wt % |
| IL1a level on | 168 | 119 | 114 | 115 | 129 | 87 |
| UVA exposure | | | | | | |
| ROS value on | 133 | 95 | 101 | 100 | 112 | 76 |
| UVA exposure | | | | | | |
| IL1a level on | 193 | 116 | Not | Not | 143 | 81 |
| UVB exposure | | | measured | measured | | |
| ROS value on | 278 | 125 | Not | Not | 180 | 72 |
| UVB exposure | | | measured | measured | | |

TABLE 5

| Values measured | Example C1 | Example C2 |
|---|---|---|
| IL1a level on | 114 | 115 |
| UVA exposure | | |
| ROS value on | 101 | 100 |
| UVA exposure | | |

The data in Table 4 above indicates that composition as per the invention (Example 1) provides vastly superior anti-inflammatory and anti-oxidant benefit in comparison to the sunscreen containing composition without the vitamins (B3 and C) i.e. Example C and other control compositions (Examples A and B). The data in Table-5 indicates that inclusion of only one vitamin (either Vitamin C in Example C1 and Vitamin B3 in Example C2) does not provide the superior benefit as compared to including both vitamins (Example 1).

Further, the compositions shown of examples A, B, C and 1 as shown in table 1 were exposed to a source of blue light using a blue light LED source with 5.7 J/cm2; and ROS generation was estimated using the DCFDA assay as described earlier. The ROS generation value obtained from untreated cells was taken as 100%. The values obtained when compositions of examples A, B, C and 1 were used, were as shown in table 6 below:

TABLE 6

| | Example | Example | Example | Example |
|---|---|---|---|---|
| Values measured | A | B | C | 1, wt % |
| ROS value when | 272 | 164 | 224 | 105 |
| exposed to blue light | | | | |
| (5.7 J/ cm²) | | | | |

Thus, the data in table 6 shows that the composition as per the present invention provides protection from oxidative stress induced by visible light, e.g. blue light.

1. A photoprotective composition comprising:
(i) 8 to 10 wt % of an oil-soluble sunscreen, wherein the oil-soluble sunscreen is selected from t-butylmethoxydibenzoylmethane, octyl salicylate, and 2-cyano-3,3-diphenyl-2-propenoate, or mixtures thereof;
(ii) 0.2 to 5 wt % of a water-soluble UVB sunscreen, wherein the water-soluble UVB sunscreen is 2-phenylbenzimidazole-5-sulfonic acid;
(iii) 0.1 to 5 wt % of a non-ionic surfactant having an HLB value in the range of 9 to 20;
(iv) 0.5 to 3 wt % of a vitamin B3 compound, a derivative or precursor thereof;
(v) 0.01 to 5 wt % of vitamin C or derivative thereof, and
(vi) 3 to 25 wt % of one or more saturated fatty acids comprising stearic acid.

2. The composition as claimed in claim 1, wherein the vitamin B3 compound is niacinamide.

3. (canceled)

4. (canceled)

5. The composition as claimed in claim 1, wherein the non-ionic surfactant has an HLB value in the range of 12 to 20.

6. The composition as claimed in claim 1, wherein the non-ionic surfactant is selected from one or more of a fatty alcohol ethoxylate, fatty acid ethoxylate, alkyl phenol ethoxylate, and polyoxyethylene sorbitan alkyl esters.

7. The composition as claimed in claim 1, wherein the composition comprises 4 to 23 wt % of stearic acid.

8. The composition as claimed in claim 1 further comprising palmitic acid.

9. The composition as claimed in claim 1 further comprising 0.4 to 10 wt % of a humectant.

10. The composition as claimed in claim 9, wherein said humectant is selected from one or more of a propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, glycerol, ethoxylated glycerol, and propoxylated glycerol.

11. The composition as claimed in claim 1 further comprising 0.25 to 5 wt % of a polymer of (a) dimethicone/vinyldimethicone crosspolymer, and (b) silica.

12. The composition as claimed in claim 1 further comprising a modified starch.

13. A method of providing photoprotection with a Sun Protection Factor (SPF) of at least 15 comprising applying the composition as claimed in claim 1 on to a skin surface.

14. A method of protecting skin against UVA and UVB induced inflammation and oxidative stress comprising applying the composition as claimed in claim 1 to skin.

15. A method of protecting skin against visible light induced oxidative stress comprising applying a composition as claimed in claim 1 to skin.

16. The composition as claimed in claim 6, wherein the non-ionic surfactant is a fatty alcohol ethoxylate or a fatty acid ethoxylate.

17. The composition as claimed in claim 1, wherein the vitamin C or derivative thereof is sodium ascorbyl phosphate.

18. A photoprotective composition comprising:
(i) 8 to 10 wt % of an oil-soluble sunscreen, wherein the oil-soluble sunscreen is t-butylmethoxydibenzoylmethane, octyl salicylate, and 2-cyano-3,3-diphenyl-2-propenoate;
(ii) 0.2 to 1.5 wt % of a water-soluble UVB sunscreen, wherein the water-soluble UVB sunscreen is 2-phenylbenzimidazole-5-sulfonic acid;
(iii) 0.1 to 5 wt % of a non-ionic surfactant having an HLB value in the range of 9 to 20, selected from a fatty alcohol ethoxylate, a fatty acid ethoxylate, or a combination thereof;
(iv) 0.5 to 1.25 wt % of niacinamide;
(v) 0.01 to 0.25 wt % of sodium ascorbyl phosphate, and
(vi) 3 to 6 wt % of stearic acid.

19. The composition as claimed in claim 18 further comprising from 1 to 10 wt % of a humectant.

20. The composition as claimed in claim 19, wherein the humectant is glycerol.

21. The composition as claimed in claim 18 further comprising a modified starch.

22. The composition as claimed in claim 21, wherein the modified starch is aluminium starch octenylsuccinate.
